(19) United States
(12) Patent Application Publication
(10) Pub. No.: US 2015/0073440 A1
(43) Pub. Date: Mar. 12, 2015

(54) SUTURE COLLECTOR

(71) Applicant: EMPIRE TECHNOLOGY DEVELOPMENT, LLC, Wilmington, DE (US)

(72) Inventor: Jiangming Li, Shijiazhuang (CN)

(73) Assignee: EMPIRE TECHNOLOGY DEVELOPMENT, LLC, Wilmington, DE (US)

(21) Appl. No.: 14/368,216

(22) PCT Filed: Dec. 11, 2011
(86) PCT No.: PCT/CN2011/084361

§ 371 Date: Nov. 26, 2014

(51) Int. Cl. A61B 17/04

(52) U.S. Cl. CPC A61B 17/0483; A61B 2017/0496
USPC 606/144

(57) ABSTRACT

A suture collector (100, 200, 300) includes a suture collecting member (111, 112, 211, 212, 311, 312, 411, 412, 511, 512, 611, 612, 711, 712) capable of releasable retaining sutures at a surgical site, and wherein the suture collector (100, 200, 300) is movable relative to the surgical site. Also disclosed herein are method for using the suture collector (100, 200, 300) and a surgical device kit comprising at least one suture collector (100, 200, 300).

### BACKGROUND

1. Field

The present disclosure relates surgical procedures and devices, for example, surgical devices for managing sutures.

2. Description of the Related Art

Sutures are widely used in many types of surgical procedures to pull severed tissues, such as muscles, nerves, and skins and tendons, away from the surgery site. Typically, multiple sutures are used in such procedures. In some instances, it can be difficult to keep the individual sutures separated and valuable time can be lost in untangling the sutures. Also, it can be difficult for surgeons to locate the proper suture pairs which are to be tied and knotted if the sutures are not properly organized. To aid the surgeons, a number of suture organizers have been developed. The most common of which is known as the Gabbay-Frater organizer. Examples of this type of device include suture organizers that are specific for heart surgery as described in U.S. Pat. Nos. 4,185,636 and 4,492,229. There is a need to provide a new and improved device for organizing and/or collecting sutures, in particular multiple sutures.

### SUMMARY

Some embodiments disclosed herein include a surgical device for managing sutures surrounding a surgical site, the device comprising a suture collecting member configured to receive and releasably hold at least two surgical sutures, and wherein the device is movable relative to the surgical site.

In some embodiments the suture collecting member comprises at least one suture holder configured to receive and releasably hold at least one surgical suture.

In some embodiments the suture holder comprises at least one extension spring. In some embodiments the suture holder comprises at least one slit or clasp.

In some embodiments the suture collecting member comprises at least two clamp arms, with at least two of the clamp arms that are movable relative to one another.

In some embodiments at least one of the clamp arms is rigid. In some embodiments at least one of the clamp arms is flexible.

In some embodiments at least two of the clamp arms are substantially parallel to each other. In some embodiments at least two of the clamp arms are pivotally connected.

In some embodiments, the suture collecting member has a shape selected from the group consisting of rectangle, triangle, C-shape, U-shape, and O-shape. In some embodiments the clamp arms are shaped so as to surround an opening configured to accommodate the surgical site.

In some embodiments the diameter of the opening is not greater than 30 cm. In other embodiments, the diameter of the opening is about 1 cm to about 10 cm. In still other embodiments, the diameter of the opening is about 50 mm to about 1 μm.

In some embodiments the surgical site is a site for intestinal anastomosis, debridement, or anastomosis of blood vessels.

In some embodiments the clamp arms are pivotally connected to one another in the hinge region.

In some embodiments the surgical device comprises two handle arms pivotally connected to one another in the hinge region, wherein each of the handle arms comprising an articulating region along an end portion thereof.

In some embodiments the angle between the handle portion and the suture collecting member is about 90° to about 180°.

In some embodiments the articulating region comprises a loop sized to accommodate a thumb or a finger.

In some embodiments the clamp arms have opposing end regions and are movable between opened and closed configurations, where the opposing end regions contact one another when the clamp arms are in the closed configuration, and where at least one clamp arm comprises at least one suture holder.

In some embodiments the surgical device comprises a locking device for securing the clamp arms in one or more locked positions between the opened and closed configurations. In some embodiments the locking device is incorporated into the handle portion.

In some embodiments the clamp arms comprise an engaging surface configured to engage a living tissue.

In some embodiments the device is made of a sterilizable material. In some embodiments the sterilizable material is selected from the group consisting of stainless steel, titanium, plastic, silicon rubber, aluminum, hydronalium, polymers, carbon fiber, composite materials composed of those above-mentioned materials.

In some embodiments the surgical device comprises a handle portion with two handle arms pivotally connected to one another in a hinge region, and each handle arm comprises an articulating region along an end portion thereof. In some embodiments the clamp portion comprises two clamp arms pivotally connected to one another in the hinge region and having opposing end regions, the clamp arms are movable between opened and closed configurations, the opposing end regions contact one another when the clamp arms are in the closed configuration, and the clamp arms are shaped so as to surround an opening formed therebetween when the clamp arms are in the closed configuration. In some embodiments the opening is configured to accommodate a site on a patient, where each of the clamp arms comprises a suture collecting member, and where the suture collecting member is configured to receive and releasably hold two or more sutures. In some embodiments the surgical device comprises a locking device for securing the clamp arms in one or more locked positions between the opened and closed configurations.

Some embodiments disclosed herein include a surgical device kit, where the kit comprises: at least one surgical device for managing sutures surrounding a surgical site, the device comprises a suture collecting member configured to receive and releasably hold at least two surgical sutures, and wherein the device is movable relative to the surgical site, at least one suture, at least one suture needle, at least one suture holder, and instructions for using the surgical device, wherein the surgical device kit is contained in a package which maintains a sterile environment for the at least one surgical device, at least one suture, at least one suture needle, and at least one suture holder.

In some embodiments, the suture collecting member comprises at least one suture holder configured to receive and releasably hold at least one surgical suture.

In some embodiments, the suture holder comprises at least one extension spring. In some embodiments, the suture holder comprises at least one slit or clasp.

In some embodiments, the suture collecting member comprises at least two clamp arms, wherein at least two of the clamp arms are moveable relative to one another.

Some embodiments disclosed herein include a method for suturing a site on a patient, where the method comprises: providing a surgical device comprising a suture collecting member, where the suture collecting member is configured to receive and releasably hold two or more surgical sutures, and where the device is movable relative to the site; positioning the device along a perimeter of the site; deploying a first suture at a first position at the site and threading a free end of the first suture into a first receiving region of the suture collecting member; deploying a second suture at a second position at the site and threading a free end of the second suture into a second receiving region of the suture collecting member; and cutting the first and second sutures at the site, such that the free ends of the first and second sutures remain releasably held by the suture collecting member.

In some embodiments, the method for suturing a site comprises providing the suture collecting member which comprises one or more suture holders, where each suture holder is configured to receive and releasably hold one or more surgical sutures.

In some embodiments, the method for suturing a site comprises providing a surgical device further comprising a surgical clamp, where the surgical clamp comprises pivotally connected clamp arms having opposing end regions, where the clamp arms are movable between opened and closed configurations, and where the opposing end regions contact one another when the clamp arms are in the closed configuration, and wherein at least one clamp arm comprises one or more suture holders.

In some embodiments the method for suturing a site further comprises moving the device along with the suture releasably held by the suture collecting member away from the patient to tighten the suture.

In some embodiments the method for suturing a site further comprises moving the surgical clamp between opened and closed configurations to tighten or loosen the suture.

In some embodiments the method for suturing a site is used at a debridement site.

In some embodiments the method for suturing a site further comprises moving the device along with the suture releasably held by the suture collecting member so that the tissue around the debridement site is pulled up to accommodate an insertion of gauze into the debridement site.

### BRIEF DESCRIPTION OF THE DRAWINGS

FIG. 1A depicts a perspective view of an embodiment of the suture collector in a closed configuration.

FIG. 1B depicts a side view of an embodiment of a suture holder.

FIG. 2 depicts a perspective view of an embodiment of the suture collector in an opened configuration.

FIG. 3 depicts a perspective view of an embodiment of the suture collector with a triangle shaped suture collecting member.

FIG. 4A depicts a perspective view of an embodiment of the clamp region and suture collecting member in a closed configuration

FIG. 4B depicts a perspective view of an embodiment of the clamp region and a slideable suture collecting member in an open position.

FIG. 5A depicts a top view of an embodiment of the suture collecting member retaining sutures at a surgical site.

FIG. 5B depicts a side view of an embodiment of a suture holder.

FIG. 6 depicts a perspective view of an embodiment of the suture collecting member having slit-type suture holders.

FIG. 7A depicts a perspective view of an embodiment of the suture collecting member having clasp-type suture holders.

FIG. 7B depicts a cross-sectional view of an embodiment of the suture collecting member having clasp-type suture holders.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Disclosed in the present application are surgical devices that can be used for managing and/or collecting sutures during a surgical procedure. In some embodiments, the surgical devices are used to manage and/or collect multiple sutures surrounding a surgical site. The present disclosure also relates to methods of suturing a site on a patient by using any of the surgical devices disclosed herein.

As described herein, the surgical device can include a suture collecting member configured to receive and hold at least two surgical sutures. In some embodiments, the suture collecting member can releasably hold at least two surgical sutures. In some embodiments, the device is movable relative to the surgical site. In some embodiments, the suture holder comprises at least one helical spring, for example an extension spring. In some embodiments, the suture holder comprises at least one slit or clasp to hold the suture. In some embodiments, the suture collecting member can include at least two clamp arms, where at least two of the clamp arms are movable relative to one another. In some embodiments, the surgical device may be made of a reusable sterilizable material, or of sterile disposable material, such as plastic, as in the case of a single-use surgical device. In some embodiments the surgical device may be sterile or it may be sterilized by a user prior to use.

Although certain embodiments of the present invention are shown and described in detail, it should be understood that various changes and modifications may be made without departing from the scope of the appended claims. The scope of the present application is in no way be limited to the number of constituting components, the materials thereof, the quantities thereof, the relative arrangement thereof, etc.

In some embodiments, a suture collector provides a user with the ability to hold multiple sutures (for example, two or more sutures) in place while performing medical procedures, such as, but not limited to, intestinal, vascular, or portacaval anastomosis procedures, wound debridement, resection of small intestines or a coarctation of the aorta, repair of indirect inguinal hernia, connecting artificial valves to the natural annulus of the heart valve, surgical implantations, or any other procedures in which holding multiple sutures in place may be advantageous. By way of a non-limiting example, a user of an embodiment of a suture collector may be a surgeon, surgical assistant, doctor, nurse, emergency medical technician, dentist, plastic surgeon, physician's assistant, or similar person. Additionally, suture collector may be positioned near a surgical site and be supported in place by resting directly on a surgical site or other object or by clamp, harness, restraint, apparatus, capable of supporting a suture collector at a surgical site.

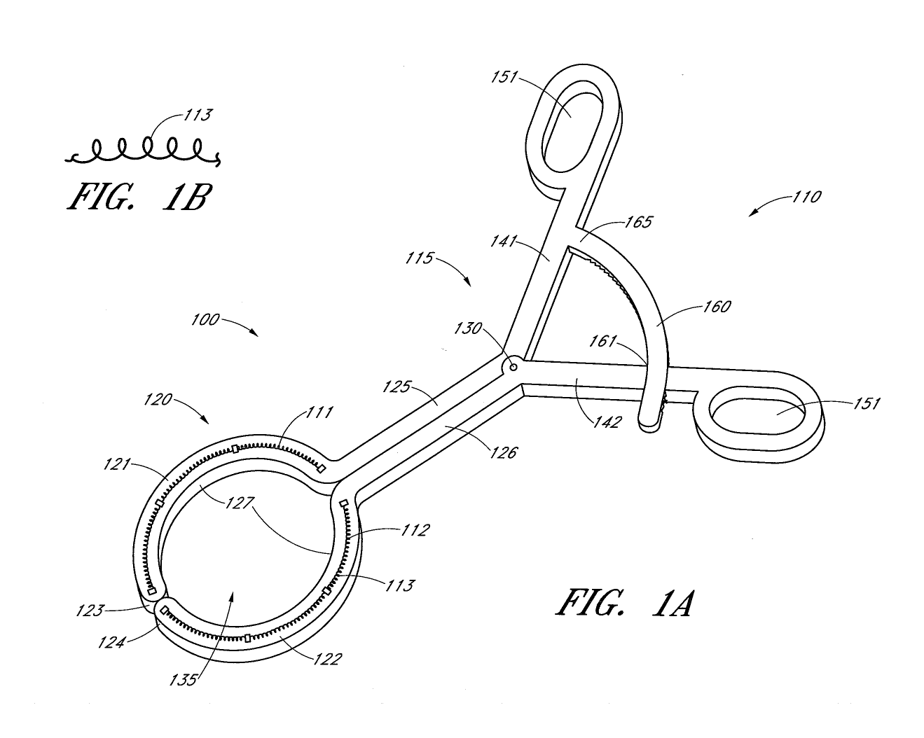

FIG. 1A illustrates a perspective view of an embodiment of a suture collector 100 in a closed configuration. Referring to FIG. 1A, a suture collector 100 comprises a handle region 110, a hinge region 115, and a clamp region 120. The handle region comprises two handles 141 and 142, which can be manipulated by a user. Handles 141 and 142 may be formed such that they form loop structures 151 which provide a user the ability to comfortably manipulate handles 141 and 142. Loop structures 151 may be shaped or sized to accommodate the fingers or thumb of a user. Skilled artisans will appreciate that any other configurations that facilitate user manipulation, besides or in addition to a loop, may be employed. In a non-limiting illustrated embodiment, handle region 110 further comprises a locking arm 160 which may be integrally formed with handle 141, or may be attached to handle 141 at an attachment point 165. The locking arm 160 may be attached at attachment point 165 by a rivet, a screw, or other similar attachment device, or may be welded, brazed, or otherwise similarly attached. Alternatively, locking arm 160 may be disposed in the clamp region 120 and may be attached thereon as described above. Locking arm 160 has a locking surface 161 which makes contact with handle 142 such that handles 141 and 142 are releasably held in place relative to each other due to the surface interactions of handle 142 and locking surface 161. Locking surface 161 may comprise one or more ratcheting teeth positioned such that the teeth releasably lock against handle 142, or locking surface 161 may comprise an engraved, marked, grooved, embossed, or similar surface so that the frictional forces created between locking surface 161 and handle 142 are sufficient to releasably lock handle 141 and 142 relative to each other.

Hinge region 115 comprises a pivot point 130 which attaches the two handles together and acts as a fulcrum when the handles are manipulated. Pivot point 130 may be a hinge, rivet, pin, bearing, or other similar structure that allows for movement of the handles relative to each other, around a fulcrum. Locking arm 160 provides the user the ability to lock handles 141 and 142 such that clamp arms 125 and 126 can be held in direct contact or close proximity at one extreme of the pivoting range, and apart from each other at the other extreme of the pivoting range.

Clamp region 120 comprises at least two clamp arms and a suture collecting member. Clamp arms 125 and 126 are individually attached to either handle 141 or 142 at pivot point 130. Clamp arms 125 and 126 may be formed of rigid material, or may be of a flexible material with sufficient resilience to provide a stable structure for supporting sutures at a surgical site. Clamp arms 125 and 126 may be integrally formed pieces with handles 141 and 142, or may be attached to handles 141 and 142 using a screw, rivet, weld, or other similar attaching method. Clamp arms 125 and 126 are individually attached to handles 141 and 142 respectively at pivot point 130 such that manipulation of handles 141 and 142 causes movement of clamp arms 125 and 126. For example, manipulation of the handles away from one another in the same plane causes clamp arms 125 and 126 to move toward one another in the same plane. Manipulation of the handles toward one another in the same plane causes clamp arms 125 and 126 to move away from one another in the same plane. It will be appreciated by skilled artisans that handles 141 and 142 may be attached at pivot point 130 to clamp arms 125 and 126 respectively in a variety of ways, not limited to that described above. Clamp arms 125 and 126 are shaped to create a void region 135 bounded by the inner engaging surface 127 of clamp arms 125 and 126 into which the target of the suture (not shown) can be placed. By way of non-limiting example, the target of the suture may be tissue at a wound site, skin, an internal body structure such as an intestine, artery, or blood vessel or other organ structure or part. Clamp arms 125 and 126 may be formed to create a void region 135 which can be ovoid, elliptical, circular, rectangular, square, teardrop, C-shaped, U-shaped, or other shape suitable for enclosing the target of the sutures. Clamp arms 125 and 126 may be sized such that the diameter of void region 135 varies according to the ideal size for a particular surgical application. For example, some embodiments of suture collector 100 may have a void region with a diameter of about 30 centimeters or less. In some embodiments the diameter of the opening is about 220 cm. In other embodiments the diameter of the opening is about 100 cm to about 200 cm. In other embodiments, the diameter of the opening is about 30 cm to 100 cm. In other embodiments, the diameter of the opening is about 10 cm to 30 cm. In other embodiments, the diameter of the opening is about 1 cm to about 10 cm. In still other embodiments, the diameter of the opening is about 50 mm to about 1 μm. These sizes are exemplary only and are not intended to limit the size of suture collector 100. Some embodiments may have a void region with a diameter of about 1 to about 10 centimeters. Some embodiments may have void regions with a diameter of about 1 micrometer to about 50 millimeters. For void region shapes which are substantially non-circular, the diameter of a void region may be measured across the longest dimension of the void region.

Suture collecting members 111 and 112 are attached to clamp arm surfaces 121 and 122 such that suture collecting members 111 and 112 may move with their respective clamp arms as handles 141 and 142 are manipulated around pivot point 130. In some embodiments, the suture collecting members can be fixedly attached to clamp arm surfaces in, e.g., disposable or reusable and sterilizable embodiments of the device. In some embodiments, the suture collecting members can be releasably attached to clamp arm surfaces, such that the suture collecting members can be replaced, e.g., where different sized interstices may be desired to accommodate different sized sutures, or where a suture collecting member may fail to provide a secure suture grip. Suture collecting members 111 and 112 may be positioned on clamp arm surfaces 121 and 122, follow the general contour of clamp arms 125 and 126, and provide an array of sites to releasably hold one or more surgical sutures. Because suture collecting members 111 and 112 generally follow the contour of clamp arms 125 and 126, the shape of suture collecting members may vary as the shape of the clamp arms varies. Suture collecting members 111 and 112 may comprise an extension spring 113 as depicted in FIG. 1B, which is formed with loops or coils wound with interstices sized to allow the extension spring 113 to act as a suture holder, releasably holding one or more surgical sutures placed into the interstices between spring windings or coils. Or, extending the extension spring 113 may allow for one or more sutures to be threaded therethrough in a tortuous path between the windings or coils of extension spring 113. Relaxing the extension spring 113, may provide a clamping action upon the sutures. Extension spring 113 can be formed of any nontoxic, noncorrosive material, such as molded plastic or stainless steel. Other configurations besides extension springs may be used as the suture holders. Suture collecting members 111 and 112 are not limited to a single suture holder 113; a plurality of suture holders can be included in the suture collecting members.

In one embodiment, the suture collector 100 may be used, for example in performing an intestinal anastomosis. The user manipulates handles 141 and 142 causing clamp arms 125 and 126 to move relative to the handles through pivot point 130. The user manipulates handles 141 and 142 toward one another in a common plane, thereby moving clamp arm tips 123 and 124 away from each other. The surgical site along the intestine for the anastomosis procedure is positioned within the void region 135 bounded by inner surface 127. Once the surgical site is within the void region 135, handles 141 and 142 are manipulated away from one another in a common plane, thereby causing clamp arm ends 123 and 124 to move toward each other into direct contact or close proximity with one another. As handles 141 and 142 are manipulated, locking surface 161 may ratchet or slide along handle 142, and once handles 141 and 142 are in the position desired by the user, locking surface 161 will provide sufficient force to hold handles 141 and 142 in the position desired. As sutures are deployed, a needle end of the suture is sown through the tissue layers to be joined and tied off, and the free end of the suture is placed or threaded into a receiving region of the suture collecting member 111 or 112 where it is releasably held by extension spring 113, thereby providing tension on the suture and support for the target of the suture while other sutures are performed. If a user desires more or less tension on the suture, handles 141 or 142 may be manipulated to widen or narrow the distance between clamp arm ends 123 and 124 thereby changing the tension on the sutures. Handles 141 and 142 are then re-locked into position via the force of the locking surface 161 on handle 142. Using suture collector 100, sutures can be placed around substantially the entirety of the target of the suture, utilizing the array of holding sites in spring coils 113 on suture collecting members 111 and 112.

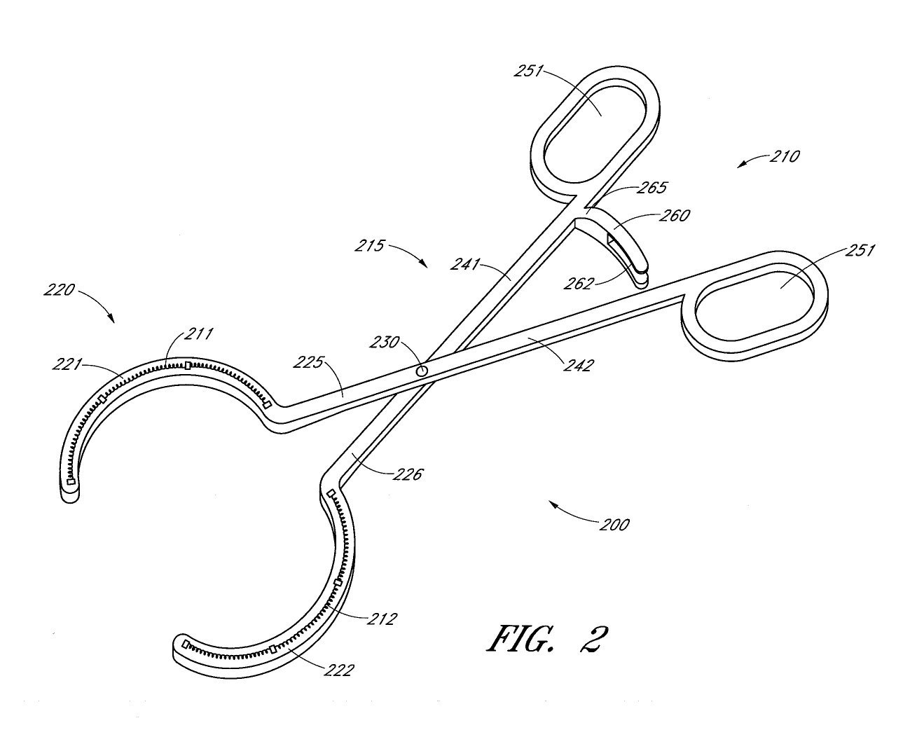

In some embodiments, the clamp arms are movable between opened and closed configurations. For example, FIG. 2 depicts a perspective view of a suture collector 200 in an opened configuration. Suture collector 200 provides a user with the ability to hold multiple sutures in place while performing medical procedures, such as, but not limited to, intestinal or vascular anastomosis procedures, wound debridement, or any other procedures in which holding multiple sutures in place may be advantageous. Referring to FIG. 2, a suture collector 200 comprises a handle region 210, a hinge region 215, and a clamp region 220. The handle region comprises two handles 241 and 242, which can be manipulated by a user. Handles 241 and 242 may be formed such that they create loop structures 251 similar to those described elsewhere herein. In the illustrated embodiment, handle region 210 further comprises a locking arm 260 which may be formed with or attached to handle 241 as described elsewhere herein. Locking arm 260 has a locking notch 262 which is sized to accept handle 242 as handles 241 and 242 are manipulated toward one another. Locking notch 262 has an interior surface which may be engraved, marked, grooved, embossed, or similarly surfaced such that handles 241 and 242 are held in place relative to each other due to the surface interactions of handle 242 and the interior surface of locking notch 262. Pivot point 230 attaches the two handles together and acts as a fulcrum when the handles are manipulated. The pivot point, as depicted, is a pin, but pivot point 230 may also be a hinge, rivet, bearing, or other similar structure that allows for movement of the handles relative to each other around a fulcrum

Clamp region 220 comprises at least two clamp arms and at least one suture collecting member. Clamp arms 225 and 226 are individually attached to either handle 241 or 242 at pivot point 230. Clamp arms 225 and 226, by way of example, are depicted as integrally formed pieces with handles 241 and 242. Clamp arms 225 and 226 may also be attached to handles 241 and 242 as described elsewhere herein. Clamp arm 225 is attached to handle 242 and clamp arm 226 is attached to handle 241 at pivot point 230 such that manipulation of handles 241 and 242 causes movement of clamp arms 225 and 226. For example, manipulation of the handles toward one another in the same plane causes clamp arms 225 and 226 to move toward one another in the same plane. Manipulation of the handles away from one another in the same plane causes clamp arms 225 and 226 to move away from one another in the same plane. It will be appreciated by a skilled artisan that handles 241 and 242 may be attached at pivot point 230 to clamp arms 225 and 226 respectively in a variety of ways, not limited to that described above. Clamp arms 225 and 226 and suture collecting members 211 and 212 may be constructed and operate as described elsewhere herein.

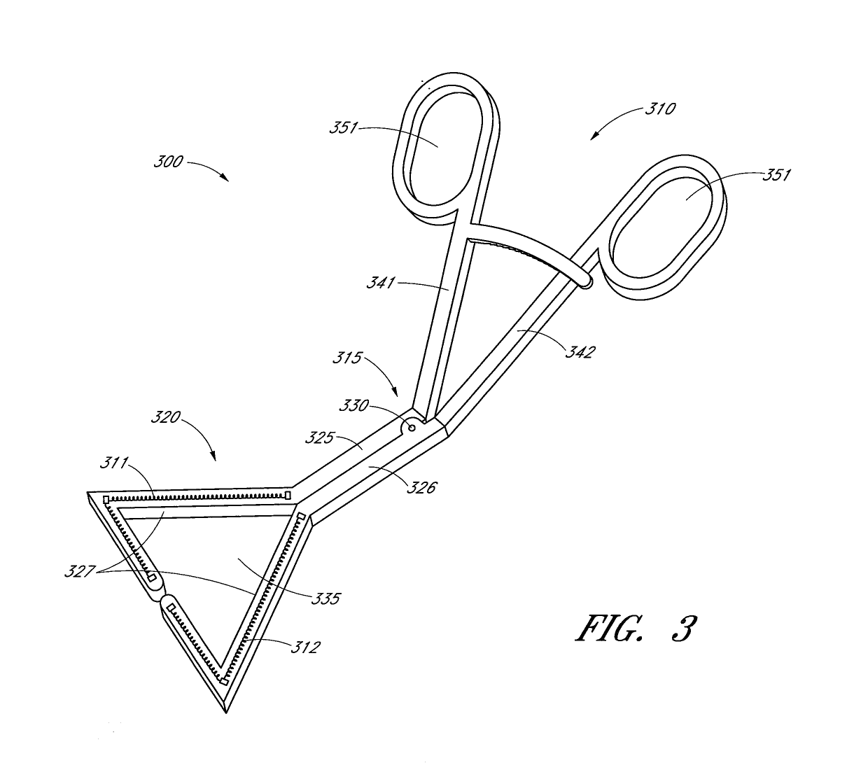

In some embodiments, the clamp arms may be of varying shapes and the handles may be disposed in a plane other than that in which the clamp arms and suture collecting members are disposed. To illustrate, FIG. 3 depicts a perspective view of a suture collector with a triangle-shaped suture collecting member. Suture collector 300 provides a user with the ability to hold multiple sutures in place while performing medical procedures, such as, but not limited to, intestinal or vascular anastomosis procedures, wound debridement, or any other procedures in which holding multiple sutures in place may be advantageous. Referring to FIG. 3, a suture collector 300 comprises a handle region 310, a hinge region 315, and a clamp region 320. The handle region comprises two handles 341 and 342, which can be manipulated by a user. Handles 341 and 342 may be formed and function as described elsewhere herein. In the illustrated embodiment, handle region 310 attaches to clamp region 320 via pivot point 330. Pivot point 330 may be constructed or operate as described elsewhere herein.

Clamp region 320 comprises two clamp arms and a suture collecting member. Clamp arms 325 and 326 are individually attached to either handle 341 or 342 at pivot point 330. Clamp arms 325 and 326 may be integrally formed pieces with handles 341 and 342, or may be attached to handles 341 and 342 as described elsewhere herein. Clamp arms 325 and 326 are individually attached to handles 341 and 342 respectively at pivot point 330 such that handles 341 and 342 extend in a plane other than that containing clamp arms 325 and 326 and suture collecting members 311 and 312. For example, as illustrated, handles 341 and 342 extend upward at an angle out of the plane in which clamp arms 325 and 326 are disposed. It will be appreciated that handle angles other than those depicted are possible, selected based on comfort of a user or ease of positioning suture collector 300 as desired at a surgical site. For example, handles 341 and 342 may extend out of the plane of clamp arms 325 and 326 at nearly any angle, for example, about 0°, about 5°, about 10°, about 15°, about 20°, about 25°, about 30°, about 35°, about 40°, about 45°, about 50°, about 60°, about 70°, about 80°, about 90°, about 100°, about 110°, about 120°, about 130°, about 140°, about 150°, about 160°, about 170°, or about 180°, or a range between any two of these values. Furthermore, although not depicted, handles 341 may extend at an angle upward from the plane wherein clamp arms 325 and 326 are disposed, and the portion of handles 341 and 342 which form loops 351 may be disposed at an angle away from the plane of handles 341 and 342, such as in a plane parallel to the plane wherein clamp arms 325 and 326 are disposed. Clamp arms 325 and 326 are shaped to create a void region 335 bounded by the inner engaging surface 327 of clamp arms 325 and 326 into which the target of the suture (not shown) can be placed. As illustrated, clamp arms 325 and 326 may be formed to create a triangular void region 335. However, it will be appreciated that the void region 335 could be ovoid, circular, rectangular, square, teardrop, or other shape suitable for enclosing the target of the sutures.

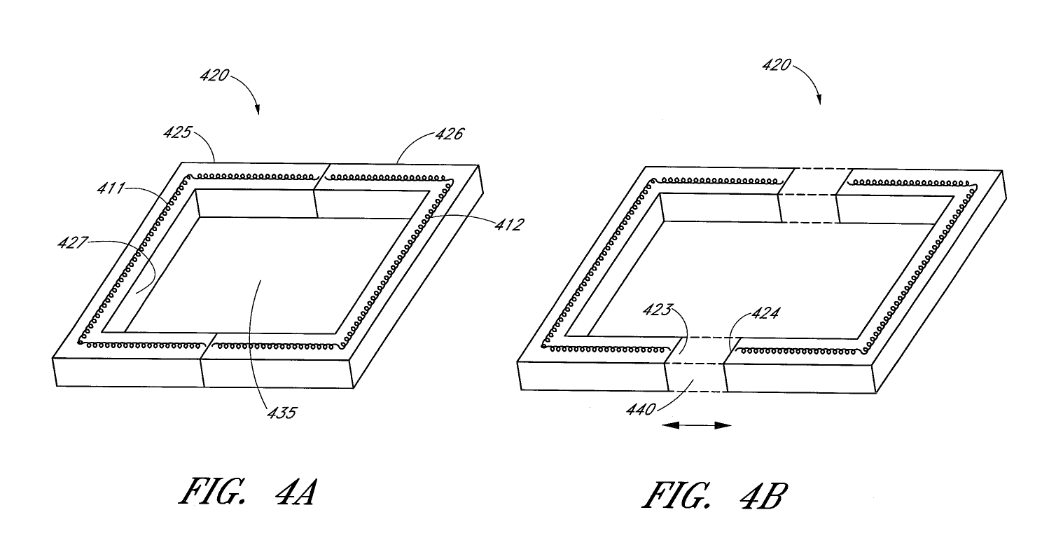

FIGS. 4A and 4B depict a perspective view of a slideable suture collecting member in a closed and open position, respectively. Referring to FIGS. 4A and 4B, the clamp arms 425 and 426 are shaped such that inner surface 427 defines a square-shaped void region 435. Clamp arms 425 and 426 may be attached to handles as described elsewhere herein. The suture collecting members 411 and 412 are positioned along clamp arms 425 and 426 as described elsewhere herein. When clamp arms 425 and 426 are manipulated, clamp arms 425 and 426 move away from each other in the same plane, along a common axis as indicated in FIG. 4B. Clamp arms 425 and 426 may be substantially hollow such that slide rail 440 is disposed within clamp arm ends 423 and 424. Slide rail 440 may have a stopping member which prevents slide rail 440 from being completely removed from clamp arm ends 423 and 424, thereby providing a maximum axial range of motion of clamp arm ends 425 and 426. The stopping member may be a tab, groove, or dimensional difference which retains stopping member within clamp arm ends 423 and 424.

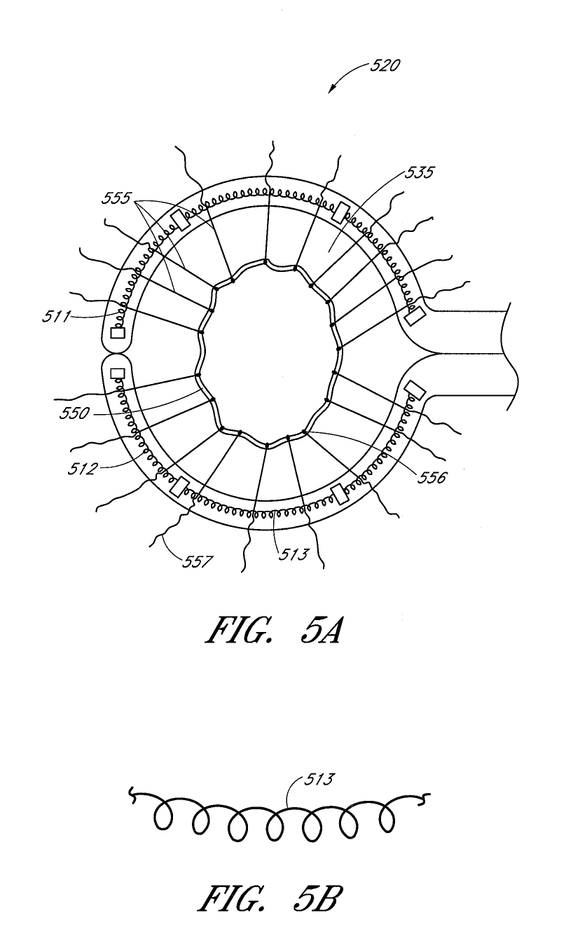

In some embodiments, the suture collecting member may be an extension spring which is able to releasably retain at least one suture or, alternatively, a plurality of sutures, as described elsewhere herein. FIG. 5 depicts a top view of a suture collecting member retaining sutures at a surgical site. Referring to FIG. 5, clamp region 520 is positioned at a surgical site. The target 550 of the suture 555 is enclosed within void region 535. Sutures 555 are deployed around the target 550. As sutures are deployed, the needle end 556 of suture 555 is sown through target 550 and tied. The free end 557 of suture 555 is placed into suture collecting member 511 or 512 where it is releasably held by extension spring 513. Extension spring 513, as depicted in FIG. 513, releasably holds and provides tension on the free end 557 of suture 555, allowing suture 555 to provide support for the target of the suture 550 while other sutures are performed. Suture collecting members 511 and 512 substantially surround target of the suture 550, enabling multiple sutures to be performed without using a multiplicity of instruments, and allowing for organized and efficient support of sutures 555 while the necessary sutures are deployed. While sutures 555 are retained within suture collecting member 511 and 512, the suture collecting members may be moved in relation to the surgical site such that sutures 555 relax or are placed under greater tension. Lifting suture collecting members 511 and 512 away from the surgical site placing the sutures under greater tension may tend to separate and organize sutures 555, preventing them from becoming tangled with each other or falling into the surgical site. Organizing sutures 555 may simplify a suturing procedure by keeping sutures 555 from tangling with each other, and allowing for easy identification of individual sutures. In some surgical application, a user desires to tie together separate sutures disposed at particular locations on the target of the suture. For example, a surgical application may require sutures which are disposed opposite or 180° from each other to be tied together. Sutures 555 which are releasably retained in suture collecting members 511 and 512 may be located and identified if cross-tying sutures are desired.

Furthermore, as suture collecting members 511 and 512 are moved away from the surgical site, tension will be created on sutures 555 disposed therein. Sutures 555 may then be sheared such that free end 557 remains releasably retained in suture collecting member 511 and 512 and needle end 556 remains in target of the suture 550.

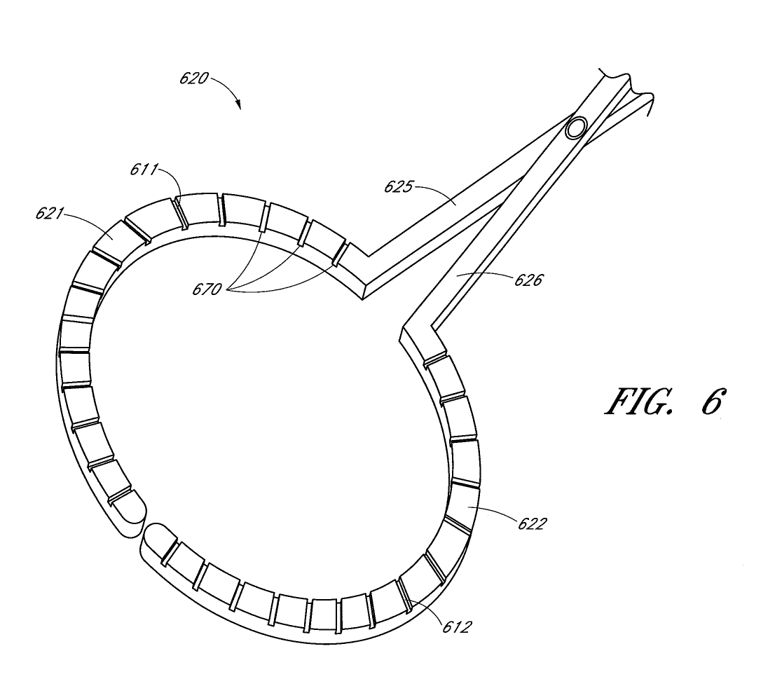

In some embodiments, the suture collecting member may comprise at least one slit which releasably retains at least one suture. For example, FIG. 6 depicts a perspective view of a suture collecting member having slit-type suture holders. Clamp region 620 may be connected via a pivot point to handles as described elsewhere herein. Suture collecting members 611 and 612 may comprise slits 670. Slits 670 are sized as to be able to releasably hold at least one surgical suture due to the frictional force of the suture and the interior of slit 670. Slits 670 may be formed as small notches in suture collecting members 611 or 612 which are in turn fixedly or releasably attached to surfaces 621 and 622 as described elsewhere herein. Alternatively, slits 670 may be formed directly into clamp arms 625 and 626 wherein there is no separate suture collecting member.

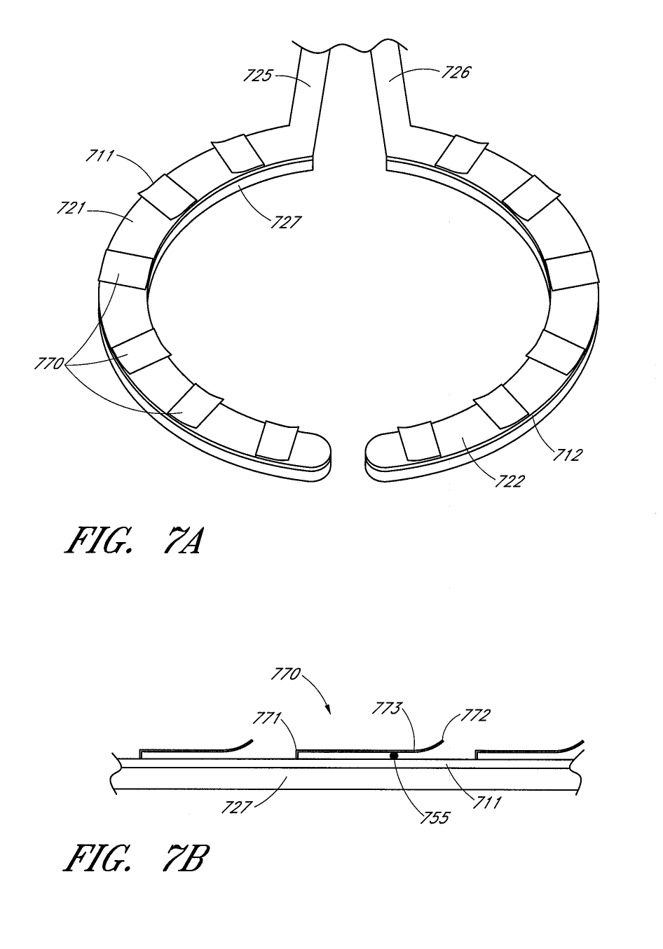

In some embodiments, the suture collecting member may comprise at least one clasp which releasably retains at least one suture. For example, FIGS. 7A and 713 depict, respectively, a perspective and cross-section view of a suture collecting member having clasp-type suture holders. Referring to FIGS. 7A and 7B, suture collecting members 711 and 712 comprise clasps 770. Referring to FIG. 7B, clasps 770 comprise a clasp bracket 773 having an attachment end 771 and a flare end 772. Attachment end 771 is attached to suture collecting member 711 or 712 such that clasp bracket 773 is disposed in close proximity to or in direct contact with surface 721 or 722 of suture collecting member 711 or 712. As suture 755 is inserted underneath clasp bracket 773, clasp bracket 773 and attachment end 771 may flex slightly, allowing entry of suture underneath the clasp bracket 773, and exerting a force on suture 755 such that suture 755 is releasably held in place due to friction between suture 755 and surface 721 or 722. Flare end 772 of clasp 770 extends upward, away from surface 721 and 722, easing the entry of surgical suture underneath clasp bracket 773. Suture collecting member 711 or 712 may be fixedly or removably attached to clamp arms 725 and 726 as described elsewhere herein. Alternatively, attachment end 771 of clasp 770 may be attached directly to the clamp arm surface with no separate suture collecting member.

In some embodiments, a suture holder may be formed by modifying a surgical clamp such as a mosquito clamp, periarticular clamp, forceps, bone repositioning forceps, bulldog clamp, or hemostatic clamp by fixedly or removably attaching a suture collecting member to the arms of the surgical clamp, attaching the suture collecting member to the clamp arms such that sutures can be supported at a surgical site where the suture holder is used.

In some embodiments, a suture holder may be formed by modifying a surgical clamp by shaping the clamp arms into a desired shape, such as those depicted elsewhere herein, e.g. ovoid, elliptical, circular, triangular, rectangular, tear-drop, U-Shaped, C-shaped, or other desired shapes, and fixedly or removably attaching a similarly shaped suture collecting member to the shaped clamp arm.

In some embodiments, the suture holder may be part of a suture holder kit. In some embodiments, the kit can comprise any suture holder as described herein, at least one suture, at least one suture needle, at least one needle holder, scissors, and forceps. The suture holder kit can also comprise a package which maintains the contents of the kit, e.g., a suture holder, at least one suture, and other components in a sterile environment and which can be opened prior to use at a surgical site. The package may comprise a variety of materials. By way of non-limiting example the package material may comprise medical-grade paper, latex-reinforced paper, polypropylene-polyethylene, spun high-density polyethylene, foil-lined paper, or any combination of these. The suture holder kit may be a disposable, single-use kit, or may be a sterilizable and resealable package. The suture holder kit may comprise a single package containing the kit components, or the kit may comprise separately and sterilely packaged items combined into a larger package which contains the kit components. The suture holder kit may also comprise instructions for using a suture holder. The instructions may be written or depicted a package insert or directly on the package, or both. In some embodiments, the instructions are in the form of a computer readable medium, such as a CD-ROM or a DVD-ROM.

### EXAMPLES

Additional embodiments are disclosed in further detail in the following examples, which are not in any way intended to limit the scope of the claims.

### Example 1

### Suture Holder Device Used During Debridement of a Necrotic Wound

This example illustrates that a suture collector as described herein can be used for wound debridement procedures.

A patient presents with a slow-healing, necrotic wound requiring debridement, and a physician chooses to employ a surgical or sharp debridement approach. The physician selects a suture holder as described herein. The physician positions the suture holder at the debridement site such that the clamp arms surround the edges of the wound. To prepare the wound for proper debridement of the necrotic tissues, the physician ties one end of a suture into the tissue surrounding the perimeter of the wound and places the free end of the suture into the suture collecting member on the suture holder. The physician ties multiple sutures around the exterior of the wound, placing the free ends of multiple sutures into the suture collecting members in an orderly fashion, where they are releasably held such that no sutures overlap and the ends of the sutures are neatly organized around the wound.

The physician then manipulates the entire suture holder away from the wound or in any desired direction in order to open the wound and facilitate debridement. The physician moves the clamp arms away from each other by manipulating the handles, thereby spreading the tissue surrounding the wound site. As the clamp arms move away from each other, the edges of the wound are pulled along with the sutures, opening the wound. This wound opening provides greater access to the necrotic tissue, bone, or internal bodily material, or any foreign objects, facilitating more complete, effective, and efficient debridement.

The physician excises the necrotic tissue or removes the foreign material, and cleans the wound. Following the excision or removal of tissue or other material from the wound, the physician then closes the wound by moving the clamp arms toward one another by manipulating the handles, and removing the free end of a suture from the suture collecting member. The physician ties the free end to a suture located at another position on the suture collecting member, such as to a suture directly across the suture collecting member. As the sutures are all tied and the wound is closed, the suture collecting member maintains organization of the individual sutures, making wound closure easier by preventing tangling of the sutures and maintaining tension on the sutures as they are tied.

### Example 2

### Suture Holder Used in Intestinal Anastomosis

This example illustrates that a suture collector as described herein can be used in intestinal anastomosis procedures.

A patient is identified who needs intestinal anastomosis after undergoing bisection or removal of a length of small intestine. A physician selects a suture holder which is appropriately shaped for the surgical site, such as a suture holder with elliptical or C-shaped clamp arms. The physician positions the appropriate suture holder at or near the site of the anastomosis. One end of the intestine which is to be reattached is inserted into the space between the clamp arms of the suture holder. The physician then ties one end of a suture into the perimeter of the intestine and attaches the free end of the suture of the suture collecting member, where it is releasably held. The physician proceeds around the perimeter of the intestine, repeating the sutures. This suturing pattern provides support for the open end of the intestine in preparation for anastomosis by allowing for easy manipulation and by positioning the intestine in a convenient and desirable orientation.

The physician manipulates the clamp arms using the handles so that the sutures releasably held in the suture collecting member pull the opening of the intestine end into a roughly uniform shape as the clamp arms move away from each other. The surgeon pulls the intestine into a substantially round or elliptical shape in order to facilitate anastomosis. The surgeon also manipulates the shape and size of the open end of the intestine by varying the length of the free end of the sutures as they are placed into the suture collecting member.

With the sutures organized substantially uniformly around the perimeter of the intestine and one end of the intestine supported in a suture holder, the other end of the intestine is positioned near the suture holder and the physician then sutures together the two ends of the intestine. Additionally, a physician may choose to support both ends of the bisected intestine in suture holders prior to anastomosis.

The above examples are given by way of illustration only, and are not intended to limit the potential uses of a suture holder. One skilled in the art will appreciate the other ways in which a suture holder could be employed during a surgical procedure.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

One skilled in the art will appreciate that, for this and other processes and methods disclosed herein, the functions performed in the processes and methods can be implemented in various orders. Furthermore, the outlined steps and operations are only provided as examples, and some of the steps and operations can be optional, combined into fewer steps and operations, or expanded into additional steps and operations without detracting from the essence of the disclosed embodiments.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

1. A surgical device for managing sutures surrounding a surgical site comprising:
a suture collecting member configured to receive and releasably hold at least two surgical sutures, the suture collecting member comprising at least two pivotally connected clamp arms, the clamp arms being movable relative to one another; and wherein the device is movable relative to the surgical site.

2. The surgical device of claim 1, wherein the suture collecting member comprises at least one suture holder configured to receive and releasably hold at least one surgical suture.

3. The surgical device of claim 2, wherein the suture holder comprises at least one extension spring.

4. The surgical device of claim 2, wherein the suture holder comprises at least one slit or clasp.

5. (canceled)

6. (canceled)

7. (canceled)

8. (canceled)

9. (canceled)

10. (canceled)

11. The surgical device of claim 1, wherein the clamp arms are shaped so as to surround an opening configured to accommodate a surgical site.

12. (canceled)

13. (canceled)

14. (canceled)

15. The surgical device of claim 11, wherein the surgical site is a site for intestinal anastomosis, debridement, or anastomosis of blood vessels.

16. (canceled)

17. The surgical device of claim 1, further comprising a handle portion comprising at least two handle arms each connected to one of the clamp arms.

18. The surgical device of claim 17, wherein the angle between the handle portion and the suture collecting member is about 90° to about 180°.

19. (canceled)

20. The surgical device of claim 1, wherein the pivotally connected clamp arms have opposing end regions and are movable between an open configuration and a closed configurations, and wherein the opposing end regions contact one another when the clamp arms are in the closed configuration.

21. The surgical device of claim 20, further comprising a locking device for securing the clamp arms in one or more locked positions between the opened and closed configurations.

22. (canceled)

23. (canceled)

24. (canceled)

25. (canceled)

26. A clamp comprising:
a handle portion comprising two handle arms pivotally connected to one another in a hinge region, each handle arm comprising an articulating region along an end portion thereof;
a clamp portion comprising two clamp arms pivotally connected to one another in the hinge region and having opposing end regions,
wherein the clamp arms are movable between opened and closed configurations,
wherein the opposing end regions contact one another when the clamp arms are in the closed configuration,
wherein the clamp arms are shaped so as to surround an opening formed therebetween when the clamp arms are in the closed configuration, and wherein the opening is configured to accommodate a site on a patient, and
wherein each of the clamp arms comprises a suture collecting member, wherein the suture collecting member is configured to receive and releasably hold two or more sutures; and
a locking device for securing the clamp arms in one or more locked positions between the opened and closed configurations.

27. (canceled)

28. (canceled)

29. The clamp of claim 26 wherein the suture collecting member comprises at least one extension spring.

30. The clamp of claim 26 wherein the suture collecting member comprises at least one slit or clasp.

31. (canceled)

32. A method for suturing a site on a patient, the method comprises:
providing a surgical device comprising a suture collecting member, wherein the suture collecting member is configured to receive and releasably hold two or more surgical sutures, and wherein the device is movable relative to the site;
positioning the device along a perimeter of the site;
deploying a first suture at a first position at the site and threading a free end of the first suture into a first receiving region of the suture collecting member;
deploying a second suture at a second position at the site and threading a free end of the second suture into a second receiving region of the suture collecting member; and
cutting the first and second sutures at the site, such that the free ends of the first and second sutures remain releasably held by the suture collecting member.

33. The method of claim 32, wherein the suture collecting member comprises one or more suture holders, wherein each suture holder is configured to receive and releasably hold one or more surgical sutures.

34. The method of claim 32, wherein the surgical device further comprises a surgical clamp, wherein the surgical clamp comprises pivotally connected clamp arms having opposing end regions, wherein the clamp arms are movable between opened and closed configurations, and wherein the opposing end regions contact one another when the clamp arms are in the closed configuration, and wherein at least one clamp arm comprises one or more suture holders.

35. The method of claim 32, wherein the method further comprises moving the device along with the suture releasably held by the suture collecting member away from the patient to tighten the suture.

36. The method of claim 35, wherein the method further comprises moving the surgical clamp between opened and closed configurations to tighten or loosen the suture.

37. The method of any one of claim 32, wherein the site is a debridement site.

38. The method of claim 37, wherein the method further comprises moving the device along with the suture releasably held by the suture collecting member so that the tissue around the debridement site is pulled up to accommodate an insertion of one or more gauze into the debridement site.
